(19) United States
(12) Patent Application Publication
(10) Pub. No.: US 2018/0268924 A1
(43) Pub. Date: Sep. 20, 2018

(54) METHODS FOR LABORATORY SAMPLE TRACKING AND DEVICES THEREOF

(71) Applicant: True Health IP LLC, Frisco, TX (US)

(72) Inventor: J. Michael Woodard, SR., Mechanicsville, VA (US)

(21) Appl. No.: 15/911,918

(22) Filed: Mar. 5, 2018

Related U.S. Application Data
(63) Continuation of application No. 14/504,120, filed on Oct. 1, 2014, now abandoned.
(60) Provisional application No. 61/885,835, filed on Oct. 2, 2013.

(51) Int. Cl. G16H 10/40; G06Q 10/08; G16H 40/20; G16H 40/63

(52) U.S. Cl. CPC G16H 10/40; G16H 40/63; G16H 40/20; G06Q 10/0833

(57) ABSTRACT

A method of managing and tracking a collected laboratory sample includes receiving a patient identifier and laboratory-workflow information related to the collected laboratory sample. Shipment information is obtained for the collected laboratory sample based on the received patient identifier. A tracking process is initiated after the shipping information has been obtained, which includes obtaining location information from one or more sample preparatory stations or sample analysis stations being used to prepare or analyze the collected laboratory sample based on the received laboratory-workflow information. Completion information related to the laboratory-workflow information is obtained indicating that the collected laboratory sample been prepared and analyzed by all designated sample preparatory stations or sample analysis stations. A status of the collected laboratory sample is provided after obtaining the shipment information, the location information, the completion information, or a combination thereof. A sample management computing device and a non-transitory computer readable medium are also disclosed.

This application is a continuation of U.S. patent application Ser. No. 14/504,120 filed Oct. 1, 2014, which claims the benefit of U.S. Provisional Patent Application Ser. No. 61/885,835 filed Oct. 2, 2013 which is hereby incorporated by reference in its entirety.

### BACKGROUND

Patient samples, such as blood samples, are often collected at one location and then shipped to a diagnostic laboratory for testing and analysis. The results are then reported electronically or by mail to the patient, as well as the physician requesting the tests. However, between shipment and reporting, the patient or physician generally do not receive further information regarding the status of the sample. Due to the serious nature of the testing performed, patients and physicians may require further information regarding the status of the collected sample. In particular, physicians may wish to monitor the status to better manage their practice and prepare treatment plans.

Existing software, such as Laboratory Information Systems (LIS) allow the internal tracking of laboratory workflows indicating when a sample is accessioned, enters into the lab, and completes the workflow. The LIS also tracks and manages the reporting process for providing the results to the requesting party. The existing software, however, does not provide external updates on the workflow with respect to specific devices and departments within the laboratory setting to provide updates related to the status of the sample to the requesting party. Further, the LIS only tracks the workflow after accession into the laboratory itself, and does not provide updates on the status of the sample throughout the lifecycle from shipment to the reporting of the results.

The present invention is directed to overcoming these and other deficiencies in the art.

### SUMMARY

One aspect of the present invention relates to a method of managing and tracking a collected laboratory sample including receiving, by a sample management computing device, a patient identifier related to the collected laboratory sample and laboratory-workflow information related to the collected laboratory sample. Shipment information is obtained for the collected laboratory sample based on the received patient identifier. A tracking process is initiated after the shipping information has been obtained. The tracking process includes obtaining, by the sample management computing device, location information from one or more sample preparatory stations or sample analysis stations being used to prepare or analyze the collected laboratory sample based on the received laboratory-workflow information. Completion information related to the laboratory-workflow information is obtained indicating that the collected laboratory sample been prepared and analyzed by all designated sample preparatory stations or sample analysis stations. A status of the collected laboratory sample is provided after obtaining the shipment information, the location information, the completion information, or a combination thereof.

Another aspect of the invention relates to a sample management computing device including a processor coupled to a memory and configured to execute programmed instructions stored in the memory, comprising receiving a patient identifier related to the collected laboratory sample and laboratory-workflow information related to the collected laboratory sample. Shipment information is obtained for the collected laboratory sample based on the received patient identifier. A tracking process is initiated after the shipping information has been obtained. The tracking process includes obtaining location information from one or more sample preparatory stations or sample analysis stations being used to prepare or analyze the collected laboratory sample based on the received laboratory-workflow information. Completion information related to the laboratory-workflow information is obtained indicating that the collected laboratory sample been prepared and analyzed by all designated sample preparatory stations or sample analysis stations. A status of the collected laboratory sample is provided after obtaining the shipment information, the location information, the completion information, or a combination thereof.

Yet another aspect of the present invention relates to a non-transitory computer readable medium having stored thereon instructions for managing and tracking a collected laboratory sample comprising machine executable code which when executed by a processor, causes the processor to perform steps including receiving a patient identifier related to the collected laboratory sample and laboratory-workflow information related to the collected laboratory sample. Shipment information is obtained for the collected laboratory sample based on the received patient identifier. A tracking process is initiated after the shipping information has been obtained. The tracking process includes obtaining location information from one or more sample preparatory stations or sample analysis stations being used to prepare or analyze the collected laboratory sample based on the received laboratory-workflow information. Completion information related to the laboratory-workflow information is obtained indicating that the collected laboratory sample been prepared and analyzed by all designated sample preparatory stations or sample analysis stations. A status of the collected laboratory sample is provided after obtaining the shipment information, the location information, the completion information, or a combination thereof.

The present technology provides a number of advantages, including providing improved methods and devices for managing and tracking a collected laboratory sample. In particular, various embodiments of the present technology provide status updates for a collected physical sample throughout the lifecycle of the workflow related to the sample, from shipment to the reporting of results. Various embodiments of the present technology further provide easy access to the status information through a web-connected portal. The present technology tracks the sample from shipment, through each step of the laboratory workflow, and through completion, and provides simple icons indicative of the status.

### BRIEF DESCRIPTION OF THE DRAWINGS

FIG. 1 is an environment including an exemplary sample management computing device.

FIG. 2 is a flowchart of an exemplary method for managing and tracking a collected laboratory sample.

### DETAILED DESCRIPTION

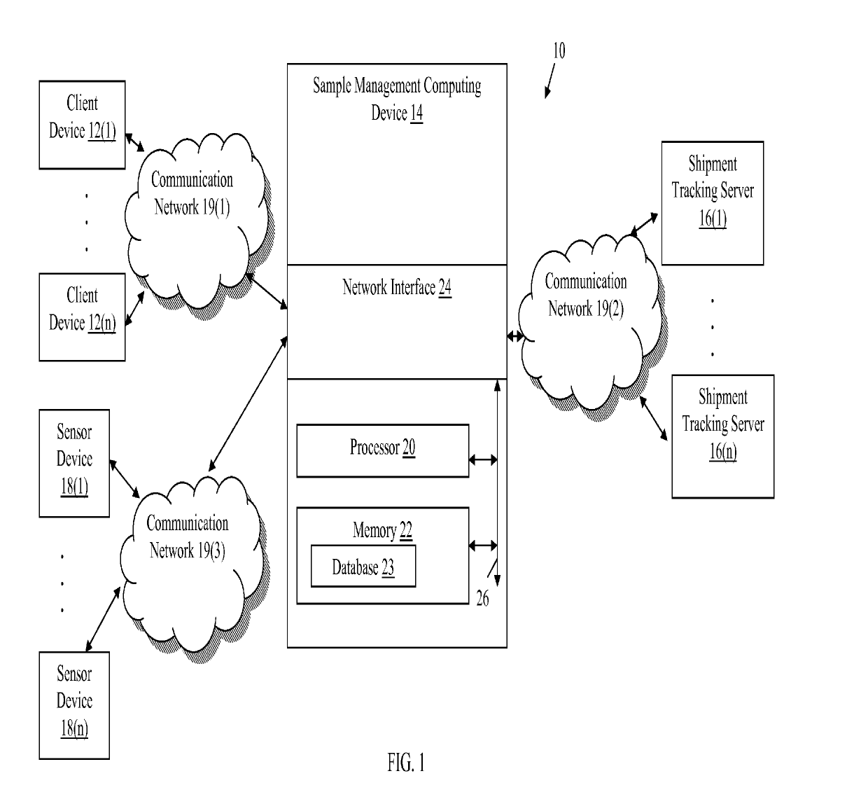

An environment 10 with an exemplary sample management computing device 14 is illustrated in FIG. 1. In this example, the environment 10 includes client devices 12(1)-12(n), the sample management computing device 14, shipment tracking servers 16(1)-16(n), and sensor devices 18(1)-18(n), which are coupled by communication networks 19(1)-19(3), although the environment could include other types and numbers of systems, devices, components, and/or other elements in other configurations and environments with other communication network topologies. This technology provides a number of advantages including providing methods, non-transitory computer readable mediums, and devices for managing and tracking a collected laboratory sample.

Referring more specifically to FIG. 1, the client devices 12(1)-12(n) enable a user to request and receive information from at least the sample management computing device 14 for the sample management and tracking illustrated and described with the examples herein, although the client devices 12(1)-12(n) could perform other types and numbers of functions and other operations. By way of example only, the client devices 12(1)-12(n) may comprise computing devices configured to communicate with the sample management computing device 14 to obtain content, such as one or more web pages, web portals, or dashboard interfaces, stored at the sample management computing device 14 and/or to send information to the sample management computing device 14.

The client devices 12(1)-12(n) include at least one processor, a memory, a user input device, a display, and a network interface, which are coupled together by a bus or other link, although one or more of client devices 12(1)-12(n) can include other numbers and types of components, parts, devices, systems, and elements in other configurations. The processor in each of the client devices 12(1)-12(n) can execute programmed instructions stored in the memory of each of the client devices 12(1)-12(n) for one or more aspects of the present invention as described and illustrated herein, although the processors could execute other numbers and types of programmed instructions.

The memory in each of the client devices 12(1)-12(n) stores these programmed instructions for one or more aspects of the present invention as described and illustrated herein, although some or all of the programmed instructions could be stored and/or executed elsewhere. A variety of different types of memory storage devices, such as a random access memory (RAM) or a read only memory (ROM) in the system or a floppy disk, hard disk, CD ROM, DVD ROM, or other computer readable medium which is read from and/or written to by a magnetic, optical, or other reading and/or writing system that is coupled to the processor, can be used for the memory in the client devices 12(1)-12(n).

The user input device in each of the client devices 12(1)-12(n) can be used to input selections, such as a request for content or information from the sample management computing device 14, although the user input devices could be used to input other types of requests and data. The user input devices can include keypads or touch screens although other types and numbers of user input devices can be used. The display in each of the client devices 12(1)-12(n) can be used to show data and information to the user, such as a dashboard interface or web portal, in response to a request for the information from the sample management computing device 14. The network interface in each of the client devices 12(1)-12(n) can be used to operatively couple and communicate between the client devices 12(1)-12(n) and at least the sample management computing device 14 via the communication network 19(1).

The sample management computing device 14 includes at least one processor 20, a memory 22, and a network interface 24, which are coupled together by a bus 26 or other link, although other numbers and types of components, parts, devices, systems, and elements in other configurations and locations can be used. The processor 20 in the sample management computing device 14 executes a program of stored instructions for one or more aspects of the present invention as described and illustrated by way of the embodiments herein, although the processor 20 could execute other numbers and types of programmed instructions.

The memory 22 in the sample management computing device 14 stores these programmed instructions for one or more aspects of the present invention as described and illustrated herein, although some or all of the programmed instructions could be stored and/or executed elsewhere. A variety of different types of memory storage devices, such as a random access memory (RAM) or a read only memory (ROM) in the system or a floppy disk, hard disk, CD ROM, DVD ROM, or other computer readable medium which is read from and/or written to by a magnetic, optical, or other reading and/or writing system that is coupled to the processor 20, can be used for the memory 22 in the sample management computing device 14.

In one embodiment, memory 20 includes database 23, although database 23 can also be stored externally and in other locations, such as by a separate network-connected server in communication with the sample management computing device 14. The database 23 can be a SQL database, by way of example, although other types of databases can be utilized. Database 23 includes patient records, which are associated with a patient identifier, such as a patient identification number, although database 23 may store other types of data.

The network interface 24 in the sample management computing device 14 is used to operatively couple and communicate between the sample management computing device 14 and the client devices 12(1)-12(n), shipment tracking servers 16(1)-16(n), and the sensor devices 18(1)-18(n) via the communication networks 19(1)-19(3), although other types and numbers of communication networks with other types and numbers of connections and configurations can be used. Additionally, the communication networks 19(1)-19(3) can include one or more local area networks (LANs) and/or wide area networks (WANs). By way of example only, the communication networks can use TCP/IP over Ethernet and industry-standard protocols, including hypertext transfer protocol (HTTP) and/or secure HTTP (HTTPS), for example, although other types and numbers of communication networks also can be used.

The shipment tracking servers 16(1)-16(n) include at least one processor, a memory, and a network interface configured to communicate with the sample management computing device 14 via communication network 19(2), although the shipment tracking servers 16(1)-16(n) could have other numbers and types of components, parts, devices, systems, and elements in other configurations and locations. Shipment tracking servers 16(1)-16(n) are communicatively coupled to various external sensors for determining the location of a shipped item, such as a laboratory sample. The location information is stored in the memory of one of the shipment tracking servers 16(1)-16(2) and may be accessed by the sample tracking computing device 14 through communication network 19(2).

The sensor devices 18(1)-18(n) include at least one processor, a memory, an input device, and a network interface configured to communicate with the sample management computing device 14 via communication network 19(3), although the sensor devices 18(1)-18(n) could have other numbers and types of components, parts, devices, systems, and elements in other configurations and locations. Sensor devices 18(1)-18(n) are attached to or associated with laboratory instruments or sample preparatory or analysis stations, such as a liquid handling apparatus, a mass spectometry instrument, an immunoassay instrument, a genotyping instrument, an electrophoresis instrument, a blood preparation instrument, a clinical chemistry analyzer, an NMR instrument, a refrigerator, or combinations thereof. The input devices of the sensor devices 18(1)-18(n) can receive an input from a laboratory sample to determine the location and identity of the laboratory sample. By way of example, the input devices can include a RFID reader, a scanner, a camera, or a manual bar code reader associated with each of the one or more laboratory instruments, although outer input devices capable or receiving an input from the laboratory sample may be utilized. Sensor devices 18(1)-18(n) can communicate the location and identity of a patient sample to the sample management computing device 14 via communication network 19(3).

Although embodiments of the sample management computing device 14, the client devices 12(1)-12(n), shipment tracking servers 16(1)-16(n), and sensor devices 18(1)-18(n) are described and illustrated herein, each of the sample management computing device 14, the client devices 12(1)-12(n), shipment tracking servers 16(1)-16(n), and sensor devices 18(1)-18(n) can be implemented on any suitable computer apparatus or computing device. It is to be understood that the apparatuses and devices of the embodiments described herein are for exemplary purposes, as many variations of the specific hardware and software used to implement the embodiments are possible, as will be appreciated by those skilled in the relevant art(s).

Furthermore, each of the devices of the embodiments may be conveniently implemented using one or more general purpose computers, microprocessors, digital signal processors, and micro-controllers, programmed according to the teachings of the embodiments, as described and illustrated herein, and as will be appreciated by those ordinary skill in the art.

The embodiments may also be embodied as one or more non-transitory computer readable medium having instructions stored thereon for one or more aspects of the present invention as described and illustrated by way of the embodiments herein, as described herein, which when executed by a processor, cause the processor to carry out the steps necessary to implement the methods of the embodiments, as described and illustrated herein.

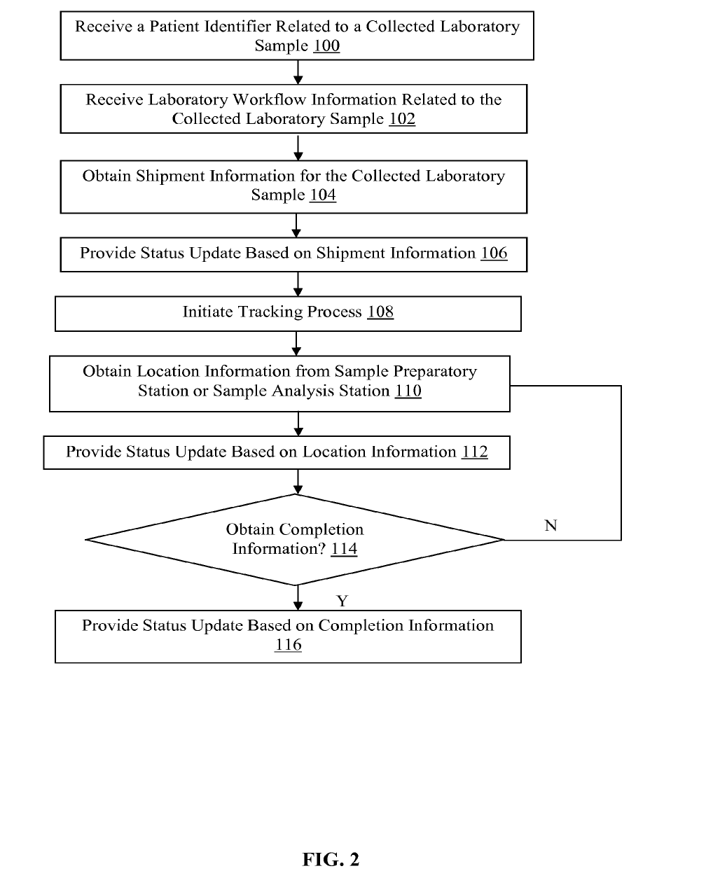

An example of a method of managing and tracking a collected laboratory sample will now be described with reference to FIGS. 1 and 2. Referring more specifically to FIG. 2, an exemplary method for managing and tracking a collected laboratory sample is illustrated. In step 100, the sample management computing device 14 receives a patient identifier related to a collected laboratory sample. By way of example, the patient identifier is a patient identification number associated with the specific patient, although other patient identifiers may be utilized to associate the patient identity with the collected sample. The patient identifier is input into one of the client devices 12(1)-12(n), for example by the physician who requested the collection of the sample, or the lab technician at the diagnostic laboratory who is processing the sample, through the user input device and is received by sample management computing device 14 via communication network 19(1), although the patient identifier may be received from other locations. The collected laboratory sample may be any physical sample associated with a patient which is further analyzed in a diagnostic laboratory, such as a blood sample, although the present method may be utilized with other physical and non-physical samples for which laboratory testing is required. In one embodiment, the collected laboratory sample is stored in a container comprising at least one of a barcode, an RFID tag, or QR code located on the container, which includes the patient identifier encoded therein.

In step 102, the sample management computing device 14 receives laboratory-workflow information related to the collected laboratory sample. The laboratory-workflow information is input into one of the client devices 12(1)-12(n), for example by the physician who requested the collection of the sample, or the lab technician at the diagnostic laboratory who is processing the sample, through the user input device and is received by sample management computing device 14 via communication network 19(1). The laboratory-workflow information indicates the diagnostic testing to be performed on the collected sample, such as testing a blood sample for markers of cardiovascular and other related diseases, although other diagnostic tests and procedures may be indicated in the laboratory-workflow information. The laboratory-workflow information may be encoded, along with the patient identifier, in the at least one of the barcode, RFID tag, or QR code located on the container holding the collected sample.

In step 104, the sample management computing device 14 obtains shipment information for the collected laboratory sample based on the received patient identifier. In one embodiment, the shipment information is received from one of the shipment tracking servers 16(1)-16(n) via communication network 19(2). The shipment information includes tracking information indicating the shipment status of the collected sample, such as en route to the diagnostic laboratory, at a shipment facility, or delivered to the diagnostic laboratory. In one embodiment, the sample management computing device 14 stores a list of shipping bills generated by the diagnostic laboratory and associated with a shipping company who maintains one of the shipment tracking servers 16(1)-16(n). The generated shipping bills may be utilized by a particular physician practice or blood draw location to ship collected samples to the diagnostic laboratory. The list of shipping bills may be stored in memory 22, although the list of generated shipping bills may be stored in other locations on other devices. The sample management computing device 14 then queries one of the shipment tracking servers 16(1)-16(n) associated with one or more of the generated shipping bills to determine whether the shipping bills have been utilized. Upon matching one of the generated shipping bills to a utilized shipping bill, the sample management computing device 14 then queries the one of the shipment tracking servers 16(1)-16(n) associated with the shipping bill to identify the collected samples included in the shipment based on the received patient identifier. In another embodiment, a shipping notification is received from one of the client devices 12(1)-12(n) associated with a laboratory sample collector, such as a physician's office or a blood draw location. After receipt of the shipment notification, the sample management computing device 14 obtains further status updates from one of the shipment tracking servers 16(1)-16(n) associated with the delivery.

Next, in step 106 the sample management computing device 14 provides a status update based on the shipment information obtained in step 104. The status update may be provided to one of the client devices 12(1)-12(n) for display thereon, for example, via a web portal or dashboard interface. Upon matching a generated shipping bill to a utilized shipping bill and identifying the patient identifier associated with a collected sample, the shipment management computing device 14 provides a status update indicating the collected sample is en route to the diagnostic laboratory. Upon receiving confirmation from one of the shipment tracking servers 16(1)-16(n) that the shipment has been delivered, the sample management computing device may provide a status update to one of the client devices 12(1)-12(n) indicating the updated status. In one embodiment, the status updates may be provided as an icon indicative of the shipment status, such as a delivery truck for an en route shipment or a package for a delivered status, although other methods may be used to provide the status updates.

In step 108, the sample management computing device 14 initiates a tracking process. In one example, upon delivery to the diagnostic laboratory, the collected sample is scanned into a Laboratory Information System (LIS), which tracks the laboratory workflow, utilizing the at least one of the barcode, RFID tag, or QR code located on the container holding the collected sample, although other methods of initiating the tracking process may be utilized. Once the tracking process is initiated, the sample management computing device 14 tracks the location of the collected sample as it proceeds through the laboratory workflow. The collected sample may further be associated with a patient record stored in database 23 based on the patient identifier associated with the collected sample.

In step 110, the sample management computing device 14 obtains location information from one or more sample preparatory stations or sample analysis stations being used to prepare or analyze the collected laboratory sample based on the received laboratory-workflow information. The location information indicates the location of the collected sample within the diagnostic laboratory and the status within the laboratory workflow. In one embodiment, the location information further includes a time stamp to indicate the time when the location information is collected. The location information may indicate a particular department, such as an autochemistry department or a genotyping department, although other departments within the diagnostic laboratory may be indicated. The location information may further indicate particular sample handling and analyzing stations associated with certain laboratory instruments within the diagnostic laboratory, such as a liquid handling apparatus, a mass spectometry instrument, an immunoassay instrument, a genotyping instrument, an electrophoresis instrument, a blood preparation instrument, a clinical chemistry analyzer, an NMR instrument, a refrigerator, or combinations thereof, although the location information may indicate other sample handling or analyzing instruments. The departments or laboratory instruments are each associated with one of the sensor devices 18(1)-18(n), such as an RFID reader, scanner, camera, or a manual bar code reader, although other sensor devices 18(1)-18(n) associated with the laboratory instruments may be utilized to provide the location information to the sample management device 14 over communication network 19(3).

By way of example only, the laboratory workflow for a collected sample may indicate NMR testing is required. The NMR instrument may be associated with one of the sensor devices 18(1)-18(n), such as a designated barcode reader. Prior to NMR testing, a barcode on the container housing the collected sample may be scanned at the barcode reader associated with the NMR instrument. The NMR instrument then provides the location information to the sample management computing device 14 via communication network 19(2).

Next, in step 112, the sample management computing device 14 provides a status update based on the location information obtained in step 110. The status update is provided for display on one of the client devices 12(1)-12(n), by way of example through a web portal. The status update includes information related to the location of the collected sample within the laboratory workflow, such as accessioning, a particular department, a sample handling or analysis station, or a particular laboratory instrument, although other information related to the location of the collected sample may be provided in the status update. The status update may be provided as an icon indicative of the location of the collected sample. By way of example only, if the collected sample is in the autochemistry department, a Beckman AU instrument may be displayed on the web portal of one of the client devices 12(1)-12(n), or if the collected sample is in preparation for genotyping, a PCR instrument may be displayed, although other icons may be utilized to represent other locations within the diagnostic laboratory.

In step 114, the sample management computing device 14 determines whether completion information indicating that the collected laboratory sample been prepared and analyzed by all designated sample preparatory stations or sample analysis stations in the laboratory workflow has been obtained. The completion information may include a generated report of the testing results. In one example, the completion information may include confirmation from one of the shipment tracking servers 16(1)-16(n) that the generated report has been delivered to a physician's office or other delivery location. In another example, the completion information may indicate that an electronic version of the generated report has been presented via a regulatory compliant secure system. If in step 114 the sample management computing device 14 determines that the completion information has not been obtained then the No branch is taken back to step 110 where the sample management computing device 14 obtains additional location information from sample preparatory stations or sample analysis stations.

If in step 114, the sample management computing device 14 determines that the completion information has been obtained, the Yes branch is taken to step 116 where the sample management computing device 14 provides a status of the collected laboratory sample related to the completion information. The status update indicates that the laboratory workflow has been completed, i.e., that the collected laboratory sample been prepared and analyzed by all designated sample preparatory stations or sample analysis stations in the laboratory workflow and that the results are ready for reporting. The status update may further indicate that the generated report has been successfully delivered, either by shipment or in electronic format. The status updated related to the completion information may be indicated by an icon indicative of the status, such as a final report icon to indicate that the report of the results has been generated.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

1. A method of managing and tracking a collected laboratory sample comprising: receiving, by a sample management computing device, a patient identifier related to the collected laboratory sample and laboratory-workflow information related to the collected laboratory sample; obtaining, by the sample management computing device, shipment information for the collected laboratory sample based on the received patient identifier; initiating, by the sample management computing device, a tracking process after the shipping information has been obtained, wherein the tracking process comprises: obtaining, by the sample management computing device, location information from one or more sample preparatory stations or sample analysis stations being used to prepare or analyze the collected laboratory sample based on the received laboratory-workflow information; obtaining, by the sample management computing device, completion information related to the laboratory-workflow information indicating that the collected laboratory sample been prepared and analyzed by all designated sample preparatory stations or sample analysis stations; and providing, by the sample management computing device, a status of the collected laboratory sample after obtaining the shipment information, the location information, the completion information, or a combination thereof.

2. The method as set forth in claim 1 wherein obtaining the shipment information comprises: receiving, by the sample management computing device, a shipping notification associated with a laboratory sample collector; obtaining, by the sample management computing device, information related to a status of the received shipping notification from a shipping provider.

3. The method as set forth in claim 1 wherein the one or more sample preparatory stations or sample analysis stations involve laboratory instruments being used to prepare or analyze the collected laboratory sample.

4. The method as set forth in claim 3 wherein the collected laboratory sample is stored in a container comprising at least one of a barcode, an RFID tag, or QR code located on the container, and further wherein the location information from the one or more laboratory instruments is obtained from an RFID reader, scanner, camera, or a manual bar code reader associated with each of the one or more laboratory instruments.

5. The method as set forth in claim 3 wherein the obtained location information further comprises a time stamp.

6. The method as set forth in claim 1 wherein the one or more laboratory instruments are selected from the group consisting of a liquid handling apparatus, a mass spectometry instrument, an immunoassay instrument, a genotyping instrument, an electrophoresis instrument, a blood preparation instrument, a clinical chemistry analyzer, an NMR instrument, a refrigerator, and combinations thereof.

7. The method as set forth in claim 1 wherein providing the status of the collected laboratory sample comprises providing an icon for display on a user device indicating one or more of the status of the collected laboratory sample, the location information, or which, if any of the sample preparatory or sample analysis stations have been completed.

8. A sample management computing device comprising: a processor coupled to a memory and configured to execute programmed instructions stored in the memory, comprising: receiving a patient identifier related to the collected laboratory sample and laboratory-workflow information related to the collected laboratory sample; obtaining shipment information for the collected laboratory sample based on the received patient identifier; initiating a tracking process after the shipping information has been obtained, wherein the tracking process comprises: obtaining location information from one or more sample preparatory stations or sample analysis stations being used to prepare or analyze the collected laboratory sample based on the received laboratory-workflow information; obtaining completion information related to the laboratory-workflow information indicating that the collected laboratory sample been prepared and analyzed by all designated sample preparatory stations or sample analysis stations; and providing a status of the collected laboratory sample after obtaining the shipment information, the location information, the completion information, or a combination thereof.

9. The device of claim 8 wherein obtaining the shipment information comprises: receiving a shipping notification associated with a laboratory sample collector; obtaining information related to a status of the received shipping notification from a shipping provider.

10. The device as set forth in claim 8 wherein the one or more sample preparatory stations or sample analysis stations involve laboratory instruments being used to prepare or analyze the collected laboratory sample.

11. The device as set forth in claim 10 wherein the collected laboratory sample is stored in a container comprising at least one of a barcode, an RFID tag, or QR code located on the container, and further wherein the location information from the one or more laboratory instruments is obtained from an RFID reader, scanner, camera, or a manual bar code reader associated with each of the one or more laboratory instruments.

12. The device as set forth in claim 11 wherein the obtained location information further comprises a time stamp.

13. The device as set forth in claim 8 wherein the one or more laboratory instruments are selected from the group consisting of a liquid handling apparatus, a mass spectometry instrument, an immunoassay instrument, a genotyping instrument, an electrophoresis instrument, a blood preparation instrument, a clinical chemistry analyzer, an NMR instrument, a refrigerator, and combinations thereof.

14. The device as set forth in claim 8 wherein providing the status of the collected laboratory sample comprises providing an icon for display on a user device indicating one or more of the status of the collected laboratory sample, the location information, or which, if any of the sample preparatory or sample analysis stations have been completed.

15. A non-transitory computer readable medium having stored thereon instructions for providing a status of a collected laboratory sample comprising machine executable code which when executed by a processor, causes the processor to perform steps comprising: receiving a patient identifier related to the collected laboratory sample and laboratory-workflow information related to the collected laboratory sample; obtaining shipment information for the collected laboratory sample based on the received patient identifier; initiating a tracking process after the shipping information has been obtained, wherein the tracking process comprises: obtaining location information from one or more sample preparatory stations or sample analysis stations being used to prepare or analyze the collected laboratory sample based on the received laboratory-workflow information; obtaining completion information related to the laboratory-workflow information indicating that the collected laboratory sample been prepared and analyzed by all designated sample preparatory stations or sample analysis stations; and providing a status of the collected laboratory sample after obtaining the shipment information, the location information, the completion information, or a combination thereof.

16. The medium as set forth in claim 15 wherein obtaining the shipment information comprises: receiving a shipping notification associated with a laboratory sample collector; obtaining information related to a status of the received shipping notification from a shipping provider.

17. The medium as set forth in claim 15 wherein the laboratory workflow comprises one or more sample preparatory or sample analysis stations.

18. The medium as set forth in claim 17 wherein the collected laboratory sample is stored in a container comprising at least one of a barcode, an RFID tag, or QR code located on the container, and further wherein the location information from the one or more laboratory instruments is obtained from an RFID reader, scanner, camera, or a manual bar code reader associated with each of the one or more laboratory instruments.

19. The medium as set forth in claim 18 wherein the obtained location information further comprises a time stamp.

20. The medium as set forth in claim 15 wherein the one or more laboratory instruments are selected from the group consisting of a liquid handling apparatus, a mass spectometry instrument, an immunoassay instrument, a genotyping instrument, an electrophoresis instrument, a blood preparation instrument, a clinical chemistry analyzer, an NMR instrument, a refrigerator, and combinations thereof.
